(19) United States
(12) Patent Application Publication
(10) Pub. No.: US 2026/0338779 A1
(43) Pub. Date: Oct. 29, 2026

(54) METHOD FOR PREDICTING THE RESPONSE TO AN INHIBITOR OF EGFR KINASE ACTIVITY

(71) Applicant: Indivumed GmbH, Hamburg (DE)

(72) Inventors: Jonathan Woodsmith, Hamburg (DE); Alexander Flohr, Hamburg (DE)

(21) Appl. No.: 19/106,012

(22) PCT Filed: Aug. 22, 2023
(86) PCT No.: PCT/EP2023/073029

§ 371 Date: Feb. 24, 2025

(30) Foreign Application Priority Data
Aug. 25, 2022 (EP) 22192093.7

(51) Int. Cl. G01N 33/5752

(52) U.S. Cl. CPC G01N 33/5752; G01N 2333/71; G01N 2333/916; G01N 2440/14

(57) ABSTRACT

The invention relates to a method to select patients that may respond to a tyrosine kinase inhibitors (TKI) treatment. The invention further relates to a diagnostic kit for the prediction of a response to an inhibitor of EGFR kinase activity. Moreover, the invention relates use of phosphorylated T59 on SHP2 as a biomarker to predict EGFR kinase activity.

The invention relates to a method to select patients that may respond to a tyrosine kinase inhibitor (TKI) treatment. The invention further relates to a diagnostic kit for the prediction of a response to an inhibitor of EGFR kinase activity. Moreover, the invention relates to the use of phosphorylated T59 on SHP2 as a biomarker to predict EGFR kinase activity.

### BACKGROUND OF THE INVENTION

Phosphorylation of specific tyrosine residues of proteins is in many cases a characteristic feature of signal transduction in cells. Enzymes capable of catalyzing such reactions are denoted tyrosine kinases, whereas tyrosine kinase inhibitors (TKI) are pharmaceutics that inhibit the activation of proteins by tyrosine phosphorylation. Growth factor receptor tyrosine kinases play a role in the etiology and progression of a variety of disorders or diseases, including, e.g., malignant diseases in humans. These receptors are anchored by means of a transmembrane domain in the membranes of cells that express them. An extracellular domain binds to the growth factor. The binding of the growth factor to the extracellular domain results in a signal being transmitted from the exterior of the cell to the intracellular kinase domain. The transduction of this signal contributes to a variety of pleiotrophic responses, which are responsible, e.g., for the induction of DNA synthesis, altered gene expression, cell growth, proliferation and differentiation, etc.

The epidermal growth factor receptor (EGFR) is a tyrosine kinase receptor and a member of HER/ErbB family that consists of four members: EGFR (HER1/ErbB1), HER2/neu (ErbB2), HER3 (ErbB3) and HER4 (ErbB 4). The receptor consists of three parts: a glycosylated extracellular ligand-binding domain, a transmembrane domain that consists of a single chain, and an intracellular protein-tyrosine kinase domain. Ligand binding to the extracellular domain causes the receptor to dimerize and activate the protein-tyrosine kinase activity. Several growth factors, including EGF and transforming growth factor-a (TGF-α) bind to the epidermal growth factor receptor. EGFRs are widely distributed at the cell surface of the mammalian epithelial cells, fibroblasts, glial cells, keratinocytes, etc. The EGFR signaling pathway plays an important role in physiological processes like cell growth, proliferation and differentiation, etc. The functional deficiency of protein tyrosine kinase, like EGFR, or the abnormality in the activity or cellular localization of the key factors in the related signaling pathways, may all lead to occurrence of tumors, diabetes, immune deficiencies and cardiovascular diseases.

Several malignancies are associated with the mutation or an increased expression of members of the ErbB family. The overexpression of EGFR and ErbB2 are closely related to cell proliferation in certain types of tumors of epithelial origin, e.g. glioblastomas (including glioblastoma multiforme), cancers of the lung (adenocarcinomas, including bronchoalveolar carcinoma (BAC) and non-small cell lung cancer (NSCLC)), as well as breast, ovary, prostrate, pancreas, bladder, colon, colorectal, kidney, head and neck cancer. The amplification and/or overexpression of the EGF receptors on the membranes of tumor cells is associated with a poor prognosis for the patients.

Such observations have stimulated investigations directed to inhibiting the function of human EGFR or HER2 as therapeutic approaches to treat cancer. For example, anti EGFR antibodies as well as anti HER2 antibodies have shown fruitful results in human cancer therapy. So far the drugs related to EGFR available in the market include (see M. L. Uribe et al., Cancer 2021, 13, 2748) Gefitinib (Iressa®), Erlotinib (Tarceva®) and Afatinib (Gilotrif®) are orally administered quinazoline derivatives that act as selective EGFR tyrosine kinase inhibitors (EGFR-TKI) and that are used in the treatment of EGFR-mutant (ERBB1-mutant) lung cancer. Lapatinib (Tykerb®) is an orally effective quinazoline derivative which is an EGFR/HER2 dual inhibitor, used in the treatment of ErbB2-overexpressing breast cancer. The afore-mentioned TKI competitively bind to the phosphorylation site of the tyrosine kinase at the intracellular segment to block the interaction between the phosphorylation site and ATP and inhibit the phosphorylation of tyrosine and a series of downstream signal transduction, and then inhibit the growth of tumor cells. The reversible EFGR inhibitors Gefitinib and Erlotinib show favorable therapeutic effects on non-small cell lung cancer patients with EGFR mutation; they can significantly prolong the progression-free survival (PFS) and overall survival (OS) of the patients. Trastuzumab, Pertuzumab, Amivantamb (Rybrevant®) and Trastuzumab-Emtansine are monoclonal antibodies which are given intravenously, target the extracellular domain and are used for the treatment of ErbB2-positive breast cancer. Trastuzumab-Emtansine is an antibody-drug conjugate that delivers a cytotoxic drug to cells overexpressing ErbB2. Cetuximab and Panitumumab are monoclonal antibodies that target ErbB1 and are used in the treatment of colorectal cancer.

However, not all subjects respond to EGFR therapy. As a consequence, patients may be exposed to the deleterious side-effects associated with EGFR-targeted therapy without its benefit.

WO2007/106432 describes a method for predicting the response of a subject having a disease or condition mediated by EGFR (e.g., a cancer of epithelial origin, such as NSCLC) to an EGFR kinase inhibitor. According to this method, the amount of phosphorylation at residues Y1068 and T1148 in EGFR is measured. An elevated level of phosphorylation at the two residues is used as an indicator that the subject is likely to be responsive to an agent that inhibits the kinase activity of EGFR. However, this method has not become established in practice. There is currently no companion diagnostic test, wherein phosphosites on the EGFR are used to select responders for an anti tumor treatment.

A further group of enzymes that together with the tyrosine kinases play an important role in signal transduction cascades are the protein tyrosine phosphatases (PTPs) that are capable of removing phosphate groups from phosphorylated tyrosine residues on proteins. A member of the PTP family is SHP2 (Src homology region 2 domain-containing phosphatase-2), an enzyme also known as protein tyrosine phosphatase non-receptor type 11 (also denoted as PTPN11). SHP2 contains two tandem Src homology-2 domains, which function as phospho-tyrosine binding domains and mediate the interaction of SHP2 with its substrates. As a member of the SH2 family, SHP2 can be activated by a variety of upstream activities to mediate phospho-tryosine signal transduction in various cellular pathways (see Z. Song et. al, Acta Pharmaceutica Sinica B, 11 (1), 2021, 13-29.).

J. Biol. Chem., Vol 270, No. 36,1995, p. 21277-21284, describes the association of SH2 domain protein tyrosine phosphatases with EGFR in human tumor cells.

In general, Western blot reagents or Western blot assays are known from prior art.

Zhang et al., Cancer Research, vol. 81, no. 11, 2021, p. 3051-3066, relates to a study of the proteome and phosphoproteome of a series of isogenic EGFR mutant lung adenocarcinoma cell lines that are either sensitive or resistant to these drugs. It was determined whether phosphorylation changes in SHP2 are associated with PI3K/AKT and MAPK signaling in TKI resistant cells. The relative quantification of phosphorylated proteins normalized to total protein expression was performed on Western blots.

Li et al., Oncology Reports, vol. 33, no. 2, 2014, p. 951-957, relates to biomarkers for predicting response to tyrosine kinase inhibitors. For determining the phosphorylation level Western blot analysis was used.

WO2007/027916 discloses phosphorylation sites identified in signal transduction proteins and pathways underlying human carcinoma. This document further provides phosphorylation-site specific antibodies and heavy-isotope labeled peptides (AQUA peptides) for the selective detection and quantification of these phosphorylated sites/proteins. The antibodies were tested by ELISA and Western blot assays.

Grimes et al, Science Signaling, vol. 11, no. 531, 2018, relates to a study of integration of protein phosphorylation, acetylation and methylation datasets to outline lung cancer signaling network. Immunoprecipitation with antibodies specific for tyrosine was used. However, it is not experimentally possible to determine the level of T59 in SHP2 using the antibodies and method referenced in this paper. Apart from T59, SHP2 has at least 7 other known threonine residues that can be phosphorylated. It not possible to use the described antibodies to determine the signal present on T59 only. It is only possible to determine the total level of threonine phosphorylation on all proteins.

This result was confirmed by the inventors of the present invention. In particular, FIGS. 4 and 5 of the present application demonstrate that neither the total phospho-threonine signal, nor the total phosphorylation signal on SHP2 function as good classifiers for EGFR activity. As such, referenced antibodies could be used to measure all phospho-threonine residues of SHP2 (PTN11) but would have worse performance as a classifier.

To sum up, the prior art documents describe potential biomarkers that are designed to be able to identify cancers that are resistant to therapy (negative selection criteria), not to identify patients that have an active EGFR signalling pathway (positive selection criteria) which is the subject of the present invention. In contrast thereto, present invention defines a novel way of defining patients who have a benefit from an EGFR therapy, and does not sub-divide this group further into responders and non-responders (see FIG. 6 of the present invention).

Furthermore, whereas prior art is not designed to define EGFR activity (and therefore positive selection criteria for anti-EGFR therapy), according to the invention it is tested whether the phosphorylation of the mentioned biomarkers HER3 and ERK1/2 can predict EGFR activity as defined through genomic mutation status. HER3 and ERK1/2 are known signaling hubs, and phosphorylation of these is dependent on multiple input signals, and they phosphorylate many output proteins. Hence, while they may be phosphorylated in EGFR active cells, they are also phosphorylated under many other conditions or in many other molecular states, and it would not be expected that they can form the basis of an accurate EGFR activity predictor. This is confirmed by the present invention (see FIGS. 4 and 5 of the present). In particular, this data show that HER3 and ERK1/2 perform very poorly compared to pT59 on PTN11.

Due to the difference in the response of patients to anti-cancer therapy, in particular a biological treatment, there is a need to find methods of predicting which treatment regimes would suit a particular patient best. In particular, it would be desirable to have a method for stratifying patients, especially for distinguishing responders from non-responders, to identify a class of subjects who may benefit from EGFR-targeted therapy.

Therefore, it is an object of the invention to provide a method for predicting the response of a subject suffering from a disease or disorder to an inhibitor of EGFR kinase activity.

In particular, it is an object of the invention to provide a method for predicting if a patient suffering from a cancer that makes him a candidate for treatment with an inhibitor of EGFR kinase activity is responsive (susceptible, amenable) to a treatment with (the administration of) an inhibitor of the EGFR kinase activity. Especially, the method is applied to patients who have a cancer of epithelial cell origin, in particular NSCLC (non-small cell lung cancer).

The method should overcome the disadvantages of the state of the art. In particular, it is an object of the invention to provide a method for predicting the response to an inhibitor of EGFR kinase activity. Current state of the art methods are based on mutation analysis, which has its biological limitations. Over active EGFR can be caused not only by mutations, but also through other mechanisms. An IHC (immunohistochemical) based method would be a good alternative and allow to examine and identify more patients that could benefit from TKI treatment.

Furthermore, it is an object of the invention to provide a method for identifying markers that can predict the response of a subject having an over-active EGFR to an inhibitor of EGFR kinase activity.

There are tumor activating mutations that can be used to identify patients that respond to TKI therapy. For example, there are EGFR mutations, which are present in a subgroup of NSCLC adenocarcinoma patients (about 15%) that can be used as indicative standard of EGFR activity, and identify patients that respond to TKI treatment.

The phosphosite used according to the invention was identified from a NSCLC tumor data set by comparison between NSCLC cases annotated with a wild-type EGFR and mutant activated EGFR. In this analysis the phosphorylation signal of phosphosites was measured through mass spectrometry. Surprisingly, the phosphorylation of the T59 amino acid on SHP2 showed the largest statistically significant effect between the two groups, and in isolation resulted a signal strong enough to clearly distinguish samples with an activated EGFR from the bulk of the cohort.

The method according to the invention has the following advantages:
The method according to the invention can determine an individual patient's drug sensitivity prior to treatment, optionally in conjunction with genomic analysis, and thus can serve as the basis for individualized targeted therapy.
Identification of NSCLC patients with activated EGFR based on measuring a p-site signal and not trough mutation testing. The phosphosite (p-site) at T59 on SHP2 can act as a biomarker.
The method allows to identify patients with activated EGFR that would be missed by classical mutation testing.
An immunohistochemistry based companion diagnostic may be a useful alternative in a routine laboratory or clinical setting.
More patients can be examined and identified that could benefit from TKI treatment.
The stability of the phosphosite employed according to the invention is not influenced by the ischemia time. Stability of p-sites are sometimes limiting the practical use, however in this case ischemia time is no limiting factor for method of the invention.

### SUMMARY OF THE INVENTION

The invention relates to a method for predicting the response of a subject suffering from a disease or disorder to an inhibitor of EGFR kinase activity, comprising the steps:
a) collecting a sample from a tumor of a subject that has a cancer of epithelial cell origin
b) determining the level of phosphorylation of at least one phosphorylation site, comprising residue T59 in the SHP2 protein;
c) comparing the level of phosphorylation of the at least one phosphorylation site, comprising residue T59, to a control level;
   wherein a significantly elevated level of phosphorylation of at least one phosphorylation site, comprising residue T59, compared to the control level indicates that the subject is likely to be responsive to an agent that inhibits the kinase activity of EGFR.

In particular, the invention relates to a method for predicting the response of a subject suffering from non-small cell lung cancer (NSCLC) to an inhibitor of EGFR kinase activity, comprising the steps:
a) collecting a sample from a tumor of a subject that having non-small cell lung cancer (NSCLC);
b) determining the level of phosphorylation of at least one phosphorylation site, comprising residue T59 in the SHP2 protein;
c) comparing the level of phosphorylation of the at least one phosphorylation site, comprising residue T59, to a control level;
   wherein a significantly elevated level of phosphorylation of at least one phosphorylation site, comprising residue T59, compared to the control level indicates that the subject is likely to be responsive to an agent that inhibits the kinase activity of EGFR.

The invention further relates to a method for selecting a subject suffering from a disease or disorder that may respond to an inhibitor of EGFR kinase activity, comprising the steps:
a) collecting a sample from a tumor of a subject that has a cancer of epithelial cell origin
b) determining the level of phosphorylation of at least one phosphorylation site, comprising residue T59 in the SHP2 protein;
c) comparing the level of phosphorylation of the at least one phosphorylation site, comprising residue T59, to a control level;
   wherein a significantly elevated level of phosphorylation of at least one phosphorylation site, comprising residue T59, compared to the control level indicates that the subject is likely to be responsive to an agent that inhibits the kinase activity of EGFR.

In particular, the invention further relates to a method for selecting a subject suffering from non-small cell lung cancer (NSCLC) that may respond to an inhibitor of EGFR kinase activity, comprising the steps:
a) collecting a sample from a tumor of a subject that has non-small cell lung cancer (NSCLC);
b) determining the level of phosphorylation of at least one phosphorylation site, comprising residue T59 in the SHP2 protein;
c) comparing the level of phosphorylation of the at least one phosphorylation site, comprising residue T59, to a control level;
   wherein a significantly elevated level of phosphorylation of at least one phosphorylation site, comprising residue T59, compared to the control level indicates that the subject is likely to be responsive to an agent that inhibits the kinase activity of EGFR.

The invention further relates to a method for identifying over-active EGFR in a subject, comprising the steps:
i) collecting a sample from a tumor of a subject having a cancer of epithelial cell origin;
ii) determining the level of phosphorylation of at least one phosphorylation site comprising residue T59 in the SHP2 protein;
iii) comparing the level of phosphorylation of at least one phosphorylation site comprising residue T59 to a control level;
   wherein a significantly elevated level of phosphorylation of at least one phosphorylation site comprising residue T59 compared to the control level indicates that the subject has likely an over-active EGFR.

In particular, the invention further relates to a method for identifying over-active EGFR in a subject, comprising the steps:
i) collecting a sample from a tumor of a subject having non-small cell lung cancer (NSCLC);
ii) determining the level of phosphorylation of at least one phosphorylation site comprising residue T59 in the SHP2 protein;
iii) comparing the level of phosphorylation of at least one phosphorylation site comprising residue T59 to a control level;
   wherein a significantly elevated level of phosphorylation of at least one phosphorylation site comprising residue T59 compared to the control level indicates that the subject has likely an over-active EGFR.

The invention also relates to a kit for predicting the response of a subject suffering from a disease or disorder to an inhibitor of EGFR kinase, comprising means for determining the level of phosphorylation of at least one phosphorylation site, comprising residue T59 in the SHP2 protein, optionally in one or more containers.

In particular, the invention also relates to a kit for predicting the response of a subject suffering from non-small cell lung cancer (NSCLC) to an inhibitor of EGFR kinase, comprising means for determining the level of phosphorylation of at least one phosphorylation site, comprising residue T59 in the SHP2 protein, optionally in one or more containers.

The invention also relates to a kit for predicting an over-active EGFR of a subject suffering from a disease or disorder to an inhibitor of EGFR kinase, comprising means for determining the level of phosphorylation of at least one phosphorylation site, comprising residue T59 in the SHP2 protein, optionally in one or more containers.

In particular, the invention also relates to a kit for predicting an over-active EGFR of a subject suffering from non-small cell lung cancer (NSCLC) to an inhibitor of EGFR kinase, comprising means for determining the level of phosphorylation of at least one phosphorylation site, comprising residue T59 in the SHP2 protein, optionally in one or more containers.

The invention also relates to a use of phosphorylated T59 on SHP2 as a marker that can predict the response of a subject having an over active-EGFR to an inhibitor of EGFR kinase activity. This use comprises determining amino acid residues in one or more proteins from the EGFR signaling pathway that are over-or under-phosphorylated, compared to a control level.

In particular, the invention also relates to a use of phosphorylated T59 on SHP2 as a marker that can predict the response of a subject having an over active-EGFR to an inhibitor of EGFR kinase activity, comprising determining the phosphorylation of T59 on SHP2 in one or more proteins from the EGFR signaling pathway that are over-or under-phosphorylated, compared to a control level.

Another embodiment of the invention relates to a method for predicting, whether a patient suffering from non-small cell lung cancer (NSCLC) is suitable for anti-EGFR therapy, comprising the steps:
a) collecting a sample from a tumor of a subject that having non-small cell lung cancer (NSCLC);
b) determining the level of phosphorylation of at least one phosphorylation site, comprising residue T59 in the SHP2 protein;
c) comparing the level of phosphorylation of the at least one phosphorylation site, comprising residue T59, to a control level;
   wherein a significantly elevated level of phosphorylation of at least one phosphorylation site, comprising residue T59, compared to the control level indicates that the subject is suitable for anti-EGFR therapy.

In the following description of suitable and preferred embodiments of the invention the term “predicting the response of a subject suffering from a disease, in particular non-small cell lung cancer (NSCLC)” can be replaced by the term “predicting, whether a patient is suitable for anti-EGFR therapy”, if nothing specific is defined.

### DESCRIPTION OF THE INVENTION

In the sense of the invention SHP2 and PTPN11 is used synonymously.

“Protein” is used interchangeably with polypeptide, and includes protein fragments and domains as well as whole proteins.

“Phosphorylatable amino acid” or phosphorylated means any amino acid that is capable of being modified by addition of a phosphate group, and includes both forms of such amino acid.

“Phosphorylatable peptide sequence” means a peptide sequence comprising at least one phosphorylatable amino acid.

In the sense of the invention the term “subject” refers to any human or animal. A (non-human) animal includes all vertebrates, e.g. mammals and non-mammals, including cows, sheep, pigs, goats, horses, poultry, dogs, cats, non-human primates, rodents etc. In one embodiment, the subject is a human subject.

Epidermal growth factor (EGF) is a protein that stimulates cell growth and differentiation. The EGF-receptor (EGFR) binds to EGF and forms a protein-ligand interaction.

The terms “p-site”, “phosphosite” and “phosphorylation site” are used synonymously.

A first embodiment of the invention is a method for prediction of the response of a subject suffering from a disease or disorder, in particular non-small cell lung cancer, to an inhibitor of EGFR kinase activity.

A special embodiment of the invention is a method for predicting if a (human) patient suffering from a cancer that makes him a candidate for treatment with an inhibitor of EGFR kinase activity is responsive to a treatment with an inhibitor of the EGFR kinase activity, comprising the steps a) to c), as defined above and in the following, wherein phosphorylation of at least one phosphorylation site, comprising the residue T59 in the SHP2 protein, indicates that the patient is likely to be responsive to a treatment with an agent that inhibits the kinase activity of EGFR.

### Step a)

In step a) a sample from a tumor in a subject having a cancer of epithelial cell origin is collected.

Suitable samples contain tissues or cells from the tumor of a subject having a cancer of epithelial cell origin. The sample preferably comprises a biopsy sample, such as a tumor biopsy, primary tissue, metastatic tissue. In particular, the specimen can be obtained by needle biopsy, image-guided biopsy, surgical (excisional) biopsy, shave/punch biopsy, endoscopic biopsy, laparoscopic biopsy and combinations thereof.

The term “collecting sample from a tumor” as used herein, refers to a sample obtained from a patient for diagnostic aims. The tumor sample can be obtained from the patient by routine measures known to the person skilled in the art, i.e., biopsy (taken by aspiration or punctuation, excision or by any other surgical method leading to biopsy or resected cellular material). In this way, it is possible to obtain tissue for the method of the present invention. However, the tumor will not be removed (completely) during collection of a sample. In the sense of the present invention, “collecting sample from a tumor” is not intended to be directed a therapeutic treatment method.

In a preferred embodiment, the subject has an over-active EGFR.

Over-activity of EGFR is not synonymous to over-expression of EGFR. Over-active of epidermal growth factor receptor (EGFR) is observed in many cancers, sometimes accompanied by gene amplification. In other words over-active EGFR refers to an increase in EGFR-driven phosphorylation-activity, not in the EGFR expression itself.

Samples are classified as EGFR over-active based on their somatic mutation profile. In particular, a sample is stated EGFR over-active if it carries at least one activating mutations as defined below.

The over-active EGFR is caused by a mutation in EGFR or by a protein interaction with EGFR such that the mutation or the protein interaction causes the receptor to be conformationally altered and to become engaged in an active signaling state contributing to a disease condition.

In a first preferred embodiment, the over-active of EGFR is caused by a mutation in the EGFR gene. The EGFR gene is present on chromosome 7p11.2 and has 28 exons coding for a transmembrane receptor protein of 464 amino acids. Within the EGFR gene, exons 5-7 and 13-16 code for the ligand binding domain, exons 18-24 code for the tyrosine kinase domain and autophosphorylation occurs in the region encoded by exons 25-28. In particular the mutation affects at least one of the afore-mentioned domains. In patients with lung cancer that are potential candidates for a positive response to TKI therapy, EGFR mutations are in particular observed in exons 18-21.

The EGFR mutation is especially selected from a mutation that alters L858, T790, G719, L861, S768; a deletion in exon19;

In particular, the mutation includes:
the point mutant L858R,
the point mutant T790M,
the point mutant G719A/C,
the point mutant L861Q,
the point mutant S7681,
   and comparable mutations, e.g.
exon 19 deletions.

Other comparable mutations include mutations in the functional moiety in which L858 is located, such as mutations in the tyrosine kinase moiety of the EGFR, or in or near the ATP-binding pocket of EGFR.

A second preferred embodiment the over-active EGFR is caused by a protein interaction with EGFR such that the receptor to be conformationally altered and to become engaged in an active signaling state contributing to a disease condition.

Preferably, the disease or disorder from which the subject is suffering is cancer, in particular cancer of epithelial cell origin. This cancer includes primary cancer and secondary (metastatic) disease.

In particular the cancer is selected from glioblastoma, melanoma, and lung, breast, ovary, prostrate, stomach, pancreas, bladder, head, neck, colon, colorectal and kidney cancer.

Especially the disease or disorder is non-small cell lung cancer (NSCLC).

### Step b)

In step b) of the method according to the invention the level of phosphorylation of the phosphorylation site residue T59 in the SHP2 protein, is determined.

The level of phosphorylation can be determined by targeted mass spectrometric analysis on tumor tissue.

The determination can be also done by development of an antibody and/or IHC assay specifically targeting the p-site.

The antibodies for use in the invention may be produced by any suitable method known in the art. Such antibodies include, but are not limited to, polyclonal, monoclonal, humanized, phage display-derived antibodies or chimeric antibodies.

### Step c)

In step c) of the method according to the invention the level of phosphorylation of at least one phosphorylation site, comprising residue T59 in the SHP2 protein, is compared to a control level.

Thereby, a significantly elevated level of phosphorylation of at least one phosphorylation site, comprising residue T59, compared to the control level indicates that the subject is likely to be responsive to an agent that inhibits the kinase activity of EGFR.

Thereby, a significantly elevated level of phosphorylation of at least one phosphorylation site, comprising residue T59, compared to the control level indicates that the subject has an over-active EGFR.

The control level refers to the level of phosphorylation at a given amino acid residue of a protein, in particular on the amino acid side chain, in a non-cancerous tissue. In other words the control level refers to the level of phosphorylation at a given amino acid residue of a protein in a normal, non-tumorous tissue.

A “significantly elevated” level of phosphorylation (compared to a control level) is a level whose difference from the control level is statistically significant, using statistical methods that are appropriate and well-known in the art. For example, the phosphorylation of at least one phosphorylation site, comprising residue T59 of SHP2, in a subject having an over-active EGFR.

Methods for measuring the level of phosphorylation at an amino acid residue are conventional and routine. In general, the measurement relies on the existence of sets of antibodies that are specific for either the non-phosphorylated or the phosphorylated forms of a particular amino acid residue of interest in the context of a protein of interest (such as EGFR). Such antibodies are commercially available or can be generated routinely, using conventional procedures. In one embodiment, a synthetic peptide comprising an amino acid of interest from a protein of interest (either in the non-phosphorylated or phosphorylated form) is used as an antigen to prepare a suitable antibody. The antibody can be polyclonal or monoclonal. Antibodies are selected and verified to detect only the phosphorylated version of the protein but not the non-phosphorylated version of the native or denatured protein, and vice-versa.

Such antibodies can be used in a variety of ways. For example, one can prepare whole cell lysates from patient samples and spot them in an array format onto a suitable substrate, such as nitrocellulose strips or glass slides. Preferably, the proteins in the samples are denatured before spotting. In general, the cells are spotted at serial dilutions, such as two-fold serial dilutions, to provide a wide dynamic range. Suitable controls, such as positive controls or controls for control levels, can be included. Each array is then probed with a suitable detectable antibody, as described above, to determine and/or to quantitate which amino acid residue(s) in the various proteins of interest are phosphorylated. Methods for immuno-quantitation are conventional. Other suitable assays employing such antibodies to assess the level and/or degree of phosphorylation at a residue of interest include, e.g., Western blots, ELISA assays, immunhistochemistry, mass spectroscopy, and other conventional assays. Suitable methods include those that can detect the phosphoprotein in a very small sample (e.g. about 200 cells). Alternatively, methods can be used that are suitable for a large sample size (e.g. FFPE tissue sections).

Assays to measure the presence and/or level of phosphorylated residues can be readily adapted to high throughput formats, e.g. using robotics, if desired. The inventive method can further comprise determining the activation state of the EGFR.

That is, the presence or absence of an activating mutation can be determined in a patient sample. For example, a subject having both an activating mutation and hyper-phosphorylation at the SHP2 residues T59 can be determined in accordance with the present invention to be a responder, and thus selected for EGFR treatment.

Other conditions that are mediated by EGFR can also be subjected to a method of the invention. These conditions are selected from hyperproliferative conditions, such as cancers, precancerous conditions, metabolic disorders (e.g., diabetes), skin disorders or diseases, cardiovascular disease, hyperproliferative cell diseases or disorders, psoriasis, obesity, inflammatory airway disease, asthma, COPD, and neurological disorders. Patients suffering from those conditions can also be assayed by a method of the invention.

One aspect of the invention is a method for treating a subject suffering from a disease or disorder, in particular non-small cell lung cancer, having over-active EGFR, comprising the steps
i) measuring the level of phosphorylation at SHP2 residue T59 in a sample from the subject and,
if the levels of phosphorylation compared to a control level suggest that the subject is likely to be responsive to EGFR therapy,
ii) administering EGFR treatment to the subject (administering an effective level of an EGFR inhibitor, such as an inhibitor of EGFR kinase activity).

Preferably, the EGFR inhibitor is selected from BIBX 1382; Cetuximab (Erbitux); CI-1033 (Canertinib); EKB-569; EMD 55900; EMD 72000; Erlotinib (OSI-774; Tarceva); Gefitinib (ZD1839; Iressa); GW-2016; hR3; ICR-62; Lapatinib (GW-572016); Lavendustin A; Lavendustin B; Monoclonal Antibody E7.6.3; Panitumumab (ABX-EGF); PD 153035; PD- 168393; PKII 66; RG-13022; RG-14620; TheraCim hR3; Tyrophostins; Tyrphostin AG 490; Tyrophostin AG 494; Tyrphostin AG 825; Tyrphostin AG 1478; Tyrphostin 1; Tyrphostin 23 (RG-50810); Tyrphostin 25 (RG-50875); Tyrphostin 46; Tyrphostin 47 (RG-50864, AG-213); Tyrphostin 51; ZD-6474; a derivative thereof, or a combination thereof.

In a preferred embodiment the subject is a human patient suffering from NSCLC, and the EGFR kinase inhibitor is selected from Iressa® (gefitinib), Tarceva® (erlotinib), Vizimpro® (dacomitinib), Gilotrif® (afatinib), or mixtures thereof.

Combinations of these agents, with each other or with conventional agents, such as chemotherapeutic agents, can be used. Although compounds may be characterized as being “EGFR inhibitors,” “EGFR kinase inhibitors,” or “inhibitors of the EGFR pathway,” the present invention is not limited to the mechanism by which such agents achieve therapeutic efficacy. For example, a patient subset selected in accordance with the present invention may respond to EGFR treatment, although the mechanism of action may not be related to, or completely related to, modulation of the EGFR pathway.

There are a number of downstream proteins in the EGFR tyrosine kinase cascade that are hyper-phosphorylated at particular residues in subjects suffering from NSCLC and having an over-active EGFR (and/or in cells in culture having the mutation).

A further aspect of the invention is the treatment of a patient identified as potential responder with a pharmaceutical composition comprising at least one inhibitor of EGFR kinase as defined above in an effective amount and a pharmaceutically acceptable carrier.

Such a pharmaceutical composition can be administered to a subject having an over-active EGFR, wherein the subject is determined by a method according to the invention to be likely to be responsive to an EGFR kinase inhibitor.

The inhibitors discussed herein can be formulated into various compositions, e.g., pharmaceutical compositions, for use in therapeutic treatment methods. The pharmaceutical compositions can be assembled as a kit. Generally, a pharmaceutical composition of the invention comprises an anticancer-effective amount of the inhibitor. An “anticancer effective amount,” as used herein, is an amount that is sufficient to effect at least a therapeutic response in the individual over a reasonable time frame. For example, it can ameliorate, at least to a detectable degree, the symptoms of a cancer, or can inhibit the growth of a tumor, etc.

The composition can comprise a carrier, such as a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

One skilled in the art will appreciate that the particular formulation will depend, in part, upon the particular inhibitory agent or other chemotherapeutic agent, that is employed, and the chosen route of administration. Accordingly, there is a wide variety of suitable formulations of compositions. Formulations suitable for oral, parenteral, aerosol, transdermal, topical, or other forms of administration will be evident to the skilled person.

One skilled in the art can easily determine the appropriate dose, schedule, and method of administration for the exact formulation of the composition being used, in order to achieve the desired anti-cancer effective amount or effective concentration of the agent in the individual patient. One skilled in the art also can readily determine and use an appropriate indicator of the “effective concentration” of the compounds of the present invention by a direct or indirect analysis of appropriate patient samples (e.g., blood and/or tissues).

The dose of an inhibitory agent of the invention, or composition thereof, administered to an animal, particularly a human, should be sufficient to effect at least a therapeutic response in the individual over a reasonable time frame (an anticancer effective amount). The exact amount of the dose will vary from subject to subject, depending on the species, age, weight and general condition of the subject, the severity or mechanism of any disorder being treated, the particular agent or vehicle used, its mode of administration and the like. The dose used to achieve a desired anticancer concentration in vivo will be determined by the potency of the particular inhibitory agent employed, the pharmacodynamics associated with the agent in the host, the severity of the disease state of infected individuals, as well as, in the case of systemic administration, the body weight and age of the individual. The size of the dose also will be determined by the existence of any adverse side effects that may accompany the particular inhibitory agent, or composition thereof, employed. It is generally desirable, whenever possible, to keep adverse side effects to a minimum.

When given in combined therapy (e.g., an EGFR kinase inhibitor in conjunction with one or more inhibitors of downstream proteins in the EGFR signaling pathway), the inhibitors can be given at the same time, or the dosing can be staggered as desired. The two or more drugs can also can be combined in a composition. Doses of each can be less when used in combination than when either is used alone.

A second embodiment of the invention a method for identifying over-active EGFR in subject, comprising the steps:
i) collecting a sample from a tumor of a subject having a cancer of epithelial cell origin, in particular collecting a sample from a tumor of a subject having non-small cell lung cancer;
ii) determining the level of phosphorylation of at least one phosphorylation site comprising residue T59 in the SHP2 protein;
iii) comparing the level of phosphorylation of at least one phosphorylation site comprising residue T59 to a control level;

wherein a significantly elevated level of phosphorylation of at least one phosphorylation site comprising residue T59 compared to the control level indicates that the subject has likely an over-active EGFR.

A third embodiment of the invention is a kit for predicting the response of a subject suffering from a disease or disorder, in particular non-small cell lung cancer, to an inhibitor of EGFR kinase, comprising means for determining the level of phosphorylation of at least one phosphorylation site comprising residue T59 in the SHP2 protein, optionally in one or more containers.

For example, the kit can be useful for predicting the response of a subject suffering from a disease or disorder, in particular non-small cell lung cancer, caused by an over-active EGFR, in particular cancer of epithelial origin, comprising reagents and/or devices for measuring the level of phosphorylation of at least one phosphorylation site comprising residue T59.

Furthermore, the kit may comprise reagents and/or devices for preparing a sample such as collecting a tissue and/or excising a sample from the tissue; for spotting test samples on a suitable surface, such as nitrocellulose strips or glass slides; for performing immuno-quantitation such as labeled antibodies, or reagents for labeling antibodies; instructions for performing a method of the invention; etc.

The components of the kit may, optionally, be packaged in one or more containers.

Among other uses, kits according to the invention can be in experimental applications to identify a phosphorylation pattern that is predictive of the response of a subject to a therapeutic agent. A skilled person will recognize components of kits suitable for carrying out any of the methods of the invention.

Optionally, the kit according to the invention comprises suitable buffers; one or more containers or packaging material; and/or instructions for performing the method. The reagents of the kit may be in containers in which the reagents are stable, e.g. in lyophilized form or stabilized liquids. The reagents may also be in single use form, e.g., in single dosage form.

Companion diagnostic tests & compounds to determine e.g., whether a mutation exists and is linked to a specific treatment, can be found on the FDA database https://www.fda.gov/medical-devices/in-vitro-diagnostics/list-cleared-or-approved-companion-diagnostic-devices-in-vitro-and-imaging-tools.

A List of Cleared or Approved Companion Diagnostic Devices (In Vitro and Imaging Tools) can be found on the EMA webpage: https://www.ema.europa.eu/en/human-regulatory/post-authorisation/data-medicines-iso-idmp-standards/public-data-article-57-database.

The following table summarizes 5 CDx tests with their corresponding drugs approved by the FDA:
1. cobas EGFR Mutation Test v2 (Roche Molecular Systems, Inc.)
2. FoundationOne CDx (Foundation Medicine, Inc.)
3. ONCO/Reveal Dx Lung & Colon Cancer Assay (O/RDx-LCCA) (Pillar Biosciences, Inc.)
4. Oncomine Dx Target Test (Life Technologies Corporation)
5. therascreen EGFR RGQ PCR Kit (Qiagen Manchester, Ltd.)

A fourth embodiment of the invention is the use of T59 on SHP2 as a marker that can predict the response of a subject having an over active-EGFR to an inhibitor of EGFR kinase activity. This use comprises determining amino acid residues, in particular determining the phosphorylation of T59 on SHP2, in one or more proteins from the EGFR signaling pathway that are over-or under-phosphorylated, compared to a control level.

The invention will be illustrated further with reference to the examples that follow, without restricting the scope to the specific embodiments described. The invention includes all combinations of described and especially of preferred features that do not exclude each other.

### DESCRIPTION OF THE DRAWINGS

A measurement of >30,000 phosphosites in NSCLC patients, with an average of ~22,000 phosphosites per patient was performed, which includes many members of the EGFR signalling pathway. It has been tested whether any combination of these phosphosites could predict EGFR activity based on the mutation status, and by far the best predictor was the single phosphosite on SHP2.

FIG. 1 shows EGFR mutation frequencies. EGFR-activity is influenced by specific genetic mutations. The somatic variations that are detected in tumor samples from patients can be utilized as primary indicator for increased EGFR kinase-activity. FIG. 1 shows those 15 mutations that arise in at least two samples from a frequency counts of all 82 distinct mutations reported for EGFR throughout the samples in a NSCLC cohort. Those plots demonstrate that the tumor sample data basis used to identify promising phosphosites is representative since the well known EGFR-activating exon-19 deletions (23 times Glu746_Ala750del, 7 times Leu747_Pro753delinsSer, 3 times Leu747_Flu749del, and 2 t times Leu747_Thr571del) were found to be the most frequent mutations as prognosed by prior art sources. Followed by mutation at Leu858Arg (23 times), and mutations at Gly719 (6 times Gly719Ala, 2 times Gly 719Cys).

FIG. 2 shows log2-transformed intensities for PTPN11-T59. Based on the mutations presented in FIG. 1, it is possible to classify samples based on their EGFR activity. Samples that carry solely somatic EGFR mutations with increasing effects on kinase activity, it can be classified as “Activated EGFR”. Vice versa, samples that carry none or other mutations than EGFR-activating ones can be seen as “Wild-Type EGFR”. Based on this classification, we 435 Wild-Type EGFR samples and 54 activated EGFR samples were identified. When analyzing the log2 transformed intensities of PTN11-T59, a significant difference between both groups occurred. An example threshold of t≥16 (dashed horizontal line) shows that this phosphosite acts as suitable group separator. In other words, samples with activated EGFR commonly show PTN11-T59 intensities of over 16, while Wild-Type EGFR is below this threshold. Additionally, samples that carry multiple but only partially EGFR-activating mutations are classified as “Wild-Type EGFR” but also show high PTN11-T59 intensities above 16, which indicates that this method is able to find samples with activated EGFR, that might exceed the pure level of somatic mutation derived kinase-activity.

FIG. 3: Ischemia time impact. This figure represents the sample log2-transformed intensities of PTN 11-T59 as FIG. 2. The data is divided into ischemia time intervals of 5 minutes, i.e. intervals that describe the time that passed between tissue extraction and freezing. By this method it was possible to track if PTN11-T59 is stable over time. The difference between samples with “Wild-Type EGFR” and “Activated EGFR” stays intact over the complete time periods and none of the groups shows a notable downwards trend with increasing time.

FIGS. 4a), 4b), 4c) and 4d): PTN11-T59 biomarker according to the invention for EGFR kinase activity can be seen as threshold based binary classification where high scores are associated with elevated kinase activity. Hence, if each sample is ranked by its level of PTN11-T59 phosphorylation, samples with overactivated EGFR kinase activity should be enriched among the highest phosphorylation measurements. The better this enrichment, the higher the area under the curve (AUC) of the corresponding receiver operating characteristic (ROC) curve. The curve reflects the concordance of EGFR overactive samples as defined by genetic mutations, and those samples with a high phosphorylation signal (seen here as true positive rate TPR). The false positive rate here reflects the presence of EGFR wild-type samples for which a high phosphorylation signal is observed. The latter could indicate biological EGFR activating processes independent of the genetic mutation state of EGFR.

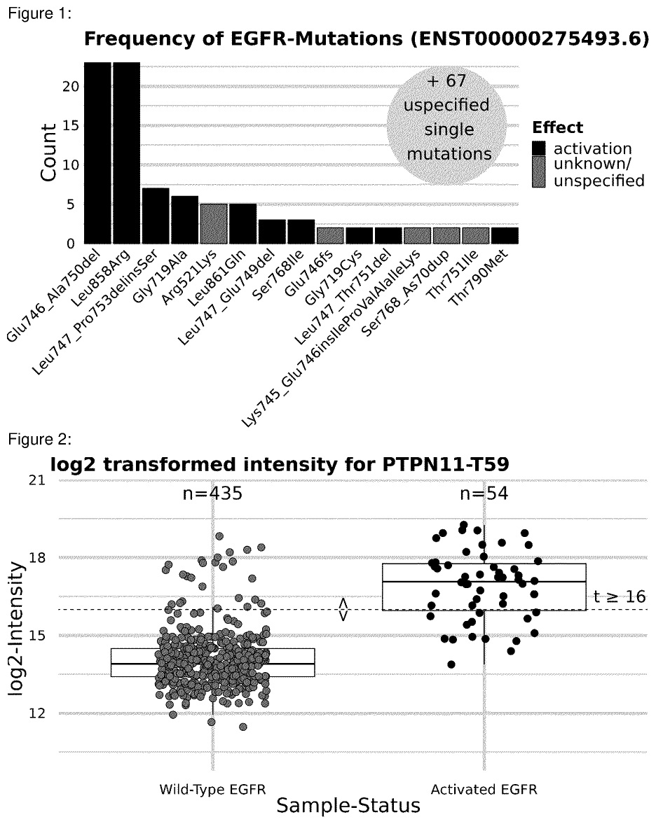

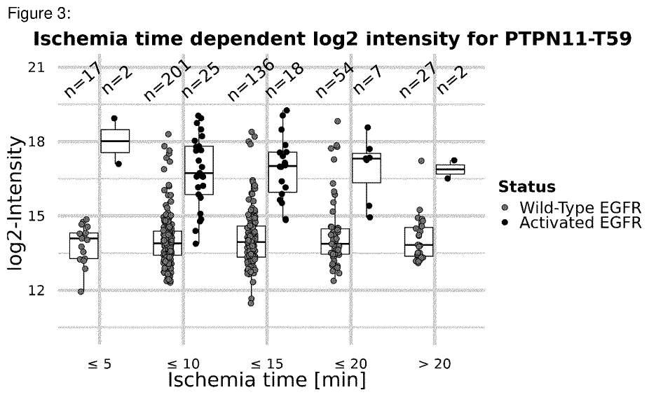

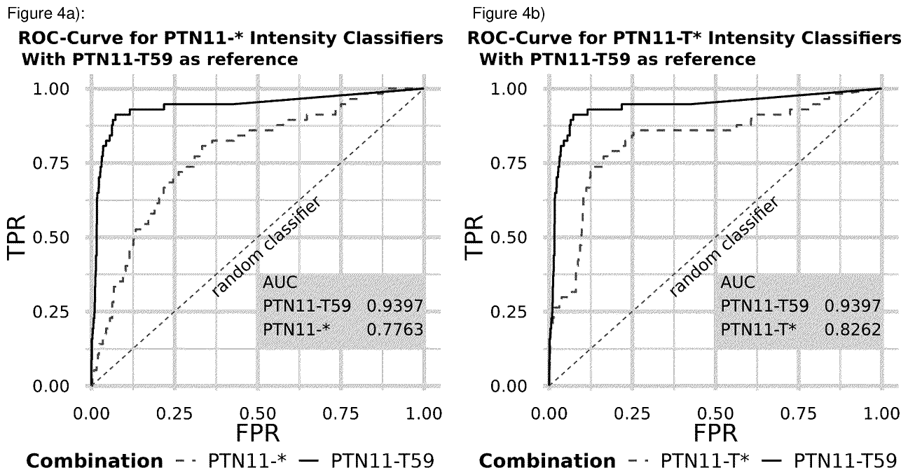

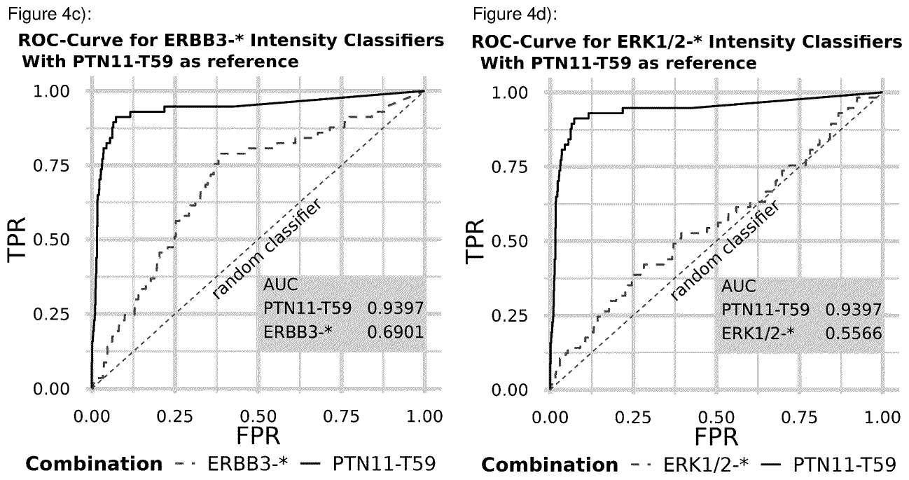

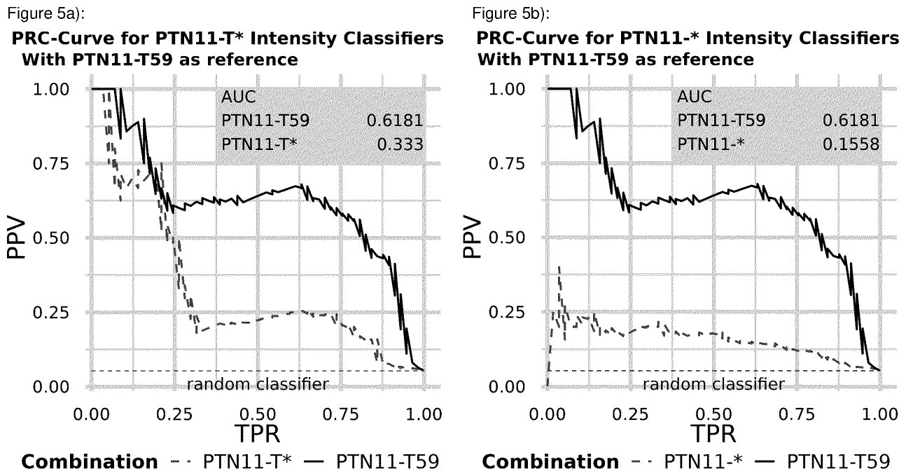

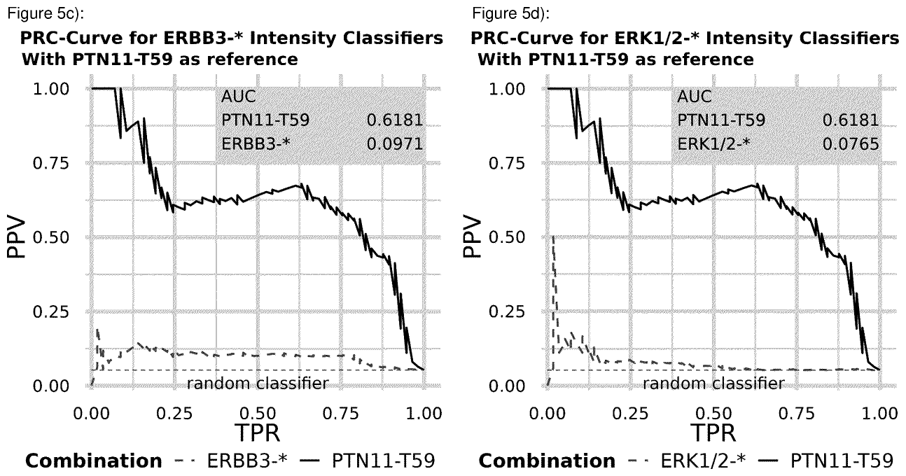

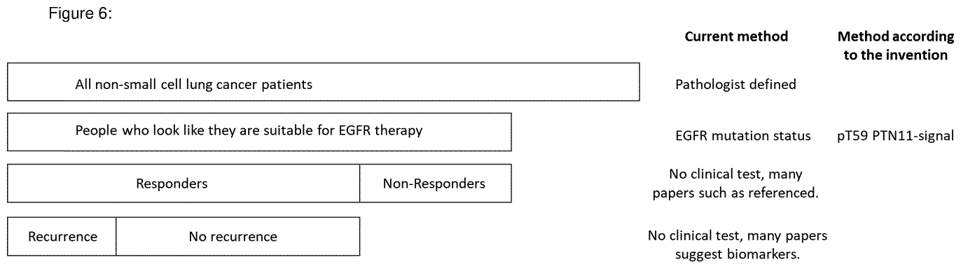

It is shown that the prediction solely based on PTN11-T59 intensities outperforms all alternative models by providing the highest AUC (area under the curve), observable through the line coming closest the top left corner of the plot, which represents the potentially best achievable model performance. Beside this, the plot further shows the decrease in performance when using a more comprehensive set of phosphosites, as the specific signal provided by PTN11-T59 is then diluted with other non-specific signals. In detail, by using the sum of phosphorylation intensities for all PTN11 threonine p-sites (PTN11-T*), the AUC drops from 0.9397 to 0.8262. It decreases even more when integrating all p-sites of PTN11 (PTN11-*) , down to 0.7763. Additionally, FIG. 4 shows that p-sites of ERBB3 (ERBB3-*) and ERK1 and ERK2 (ERK1/2-*) have a low predictive power compared to the pure PTN11-T59 model according to the invention.

Consequentially, the method to predict elevated EGFR kinase activity according to the present invention relies on a p-site-specific measurement. Hence, general protein phosphorylation is not a sufficient source of information.

FIGS. 5a), 5b), 5c) and 5d): An additional layer of information to the ROC-curves is provided by the so-called precision-recall (PR) curve. The graphic is constructed analogous to the ROC curve, but here one compares the precision (positive predictive value, PPV), the fraction of genetically defined EGFR-overactivated samples relative to the total number of samples predicted to be EGFR-overactivated using the current invention (on the y-axis) with the recall, which is equivalent to the true positive rate (TPR on the x-axis).

It is unveiled that none of the models mentioned in prior art reached an exceptionally high AUC (area under the curve) in the PR-curve plot, best is PTN11-T59 with an AUC of 0.6181. This indicates that the model according to the invention predicts previously unknown samples to be EGFR-overactivated that are expected to showcase wild-type activity based on the mutation information.

FIG. 6: A graphical representation of markers that could be useful in EGFR therapy. Firstly, patients who appear suitable for EGFR therapy are defined from all NSCLC patients. This is currently done using mutations in EGFR. According to the invention a single phosphosite on PTN11 is used to define the over activation of the EGFR. Within this group it is possible to further refine which patients may or may not respond to therapy, and furthermore which of those patients who respond to therapy may at some point get a recurrence of the tumor. Zhang et al., 2021 lists attempts to identify markers for none-responders from lung cell lines, and uses on EGFR mutations to define activity of the EGFR. Li et al., 2014 defines a negative selection criteria response to therapy. Both approaches are a negative selection criterion that has nothing to do with EGFR activity per se.

1. A method for predicting the response of a subject suffering from non-small cell lung cancer (NSCLC) to an inhibitor of EGFR kinase activity, comprising the steps:
a) collecting a sample from a tumor of a subject having;
b) determining the level of phosphorylation of at least one phosphorylation site comprising residue T59 in the SHP2 protein;
c) comparing the level of phosphorylation of at least one phosphorylation site comprising residue T59 to a control level;
wherein a significantly elevated level of phosphorylation of at least one phosphorylation site comprising residue T59 compared to the control level indicates that the subject is likely to be responsive to an agent that inhibits the kinase activity of EGFR.

2. The method according to claim 1, wherein the subject has an over-active EGFR.

3. The method according to any one of the preceding claims, wherein the over-active EGFR is caused by a mutation in EGFR or by a protein interaction with EGFR such that the mutation or the protein interaction causes the receptor to be conformationally altered and to become engaged in an active signaling state contributing to a disease condition.

4. The method according to claim 3, wherein the mutation causes the receptor to be conformationally altered and to become engaged in an active signaling state contributing to a disease condition.

5. The method according to any on of the claims 3 or 4, wherein the mutation is in a nucleic acid encoding EGFR, and is a mutation that alters L858; T790, G719, L861, S768, a deletion in exon 19.

6. The method according to any one claims 1 to 3, wherein the over-activity of EGFR is caused by a protein interaction with EGFR such that the receptor to be conformationally altered and to become engaged in an active signaling state contributing to a disease condition.

7. The method according to any one of the preceding claims, wherein the cancer includes primary and secondary metastatic disease.

8. The method according to any one of the preceding claims, wherein the subject is human.

9. The method according to any one of the preceding claims, wherein the EGFR inhibitor is BIBX 1382; Cetuximab (Erbitux); CI-1033 (Canertinib); EKB-569; EMD 55900; EMD 72000; Erlotinib (OSI-774; Tarceva); Gefitinib (ZD1839; Iressa); GW-2016; hR3; ICR-62; Lapatinib (GW-572016); Lavendustin A; Lavendustin B; Monoclonal Antibody E7.6.3; Panitumumab (ABX-EGF); PD 153035; PD-168393; PKII 66; RG-13022; RG-14620; TheraCim hR3; Tyrophostins; Tyrphostin AG 490; Tyrophostin AG 494; Tyrphostin AG 825; Tyrphostin AG 1478; Tyrphostin 1; Tyrphostin 23 (RG-50810); Tyrphostin 25 (RG-50875); Tyrphostin 46; Tyrphostin 47 (RG-50864, AG-213); Tyrphostin 51; ZD-6474; a derivative thereof, or a combination thereof.

10. The method according to any one of the preceding claims, wherein the subject is a human patient suffering from NSCLC, and the EGFR kinase inhibitor is gefitinib, erlotinib, dacomitinib, afatinib, amivantamb or mixtures thereof.

11. A method for identifying over-active EGFR in a subject, comprising the steps:
i) collecting a sample from a tumor of a subject having non-small cell lung cancer;
ii) determining the level of phosphorylation of at least one phosphorylation site comprising residue T59 in the SHP2 protein;
iii) comparing the level of phosphorylation of at least one phosphorylation site comprising residue T59 to a control level;
wherein a significantly elevated level of phosphorylation of at least one phosphorylation site comprising residue T59 compared to the control level indicates that the subject has likely an over-active EGFR.

12. A kit for predicting the response of a subject suffering from non-small cell lung cancer (NSCLC) to an inhibitor of EGFR kinase, comprising means for determining the level of phosphorylation of at least one phosphorylation site comprising residue T59 in the SHP2 protein, optionally in one or more containers.

13. A use of phosphorylated T59 on SHP2 as a marker that can predict the response of a subject having an over active-EGFR to an inhibitor of EGFR kinase activity.
